(19) United States
(12) Patent Application Publication
(10) Pub. No.: US 2023/0211126 A1
(43) Pub. Date: Jul. 6, 2023

(54) SURGICAL INSTRUMENT WITH INJECTION NEEDLE AND RETRACTABLE SHEATH

(71) Applicant: Covidien LP, Mansfield, MA (US)

(72) Inventors: Joe D. Sartor, Longmont, CO (US); Christopher T. Brown, Boulder, CO (US); Arifmohamad Hamaju Mujawar, Sangli (IN); Daniel A. Joseph, Golden, CO (US); Rajat Ravindra Rokde, Pune (IN); Sunny Kumar, Hyderabad (IN); Naveen Kanth Nelli, Ambajipeta (IN)

(21) Appl. No.: 17/926,279

(22) PCT Filed: Jun. 14, 2021
(86) PCT No.: PCT/US21/37175

§ 371 Date: Nov. 18, 2022

Related U.S. Application Data
(60) Provisional application No. 63/149,693, filed on Feb. 16, 2021.
(60) Provisional application No. 63/052,983, filed on Jul. 17, 2020.

(51) Int. Cl. A61M 25/01; A61B 34/30; A61M 25/09; A61M 25/06; A61M 39/24

(52) U.S. Cl. CPC A61M 25/0136; A61B 34/30; A61M 25/09; A61M 25/0631; A61M 39/24; A61B 2034/301

(57) ABSTRACT

A surgical instrument includes a housing (20), a syringe carriage (40), a movable handle (25), a shaft (30), a needle sheath (50), and a needle (60). The syringe carriage is operably coupled to the housing and configured to couple to a syringe. The movable handle is movable relative to the housing and operably coupled to the syringe carriage to distally advance the syringe carriage. The shaft extends distally from the housing and defines a lumen. The needle sheath extends through the lumen of the shaft, defines a lumen, and is movable relative to the shaft between an extended condition and a retracted condition. The needle extends through the lumen of the needle sheath and is configured to operably couple to a syringe coupled to the syringe carriage.

### CROSS-REFERENCE TO RELATED APPLICATIONS

This application claims the benefit of, and priority to, U.S. Provisional Application Ser. No. 63/149,693, filed on Feb. 16, 2021, and U.S. Provisional Application Ser. No. 63/052,983, filed on Jul. 17, 2020, the entire content of each of which being incorporated by reference herein.

### FIELD

This disclosure is generally related to surgical instruments and, more particularly, to surgical instruments including an injection needle and a retractable sheath for covering and uncovering a distal end of the injection needle and a surgical robotic catheter unit with an injection needle and retractable needle sheath.

### BACKGROUND

Injection of drugs in robotic procedures has been accomplished by use of a long needle catheter which is typically used with an available grasper unit. Working with these catheters is somewhat challenging as the protruding catheter needle poses safety risks to users and inadvertent tissue sticks inside the patient. An additional challenge is posed when passing the catheter needle through a laparoscopic port that contains pressure seals inside.

With growing use of indocyanine green (ICG) in near infrared (NIR) imaging as a local injection for marking or mapping tissue anatomy such as lymphatic drainage, the utility of these simple catheters becomes even more problematic. Any ICG lost from the needle will be detected by the NIR imaging camera and, as this leaked ICG spreads, its ability to mark or map tissue is lost. Additionally, providing precise injection for transversus abdominis pain (TAP) blocked under direct visualization by the surgeon provides cost effective alternative uses of ultrasound by the anesthesiologist as a separate procedure. Finally, tumescent infiltration and hydrodissection are frequently used methods to separate tissues and tissue planes using various solutions of anesthetic, epinephrine, dye, and saline. The ability to monitor pressure and total injection amounts enhances patient safety.

### SUMMARY

This disclosure is directed to a surgical instrument including an injection needle and a retractable sheath for covering and uncovering a distal end of the injection needle and a surgical robotic catheter unit with an injection needle and retractable needle sheath.

In accordance with aspects of the disclosure, a surgical instrument includes a housing, a syringe carriage, a movable handle, a shaft, a needle sheath, and a needle. The syringe carriage is operably coupled to the housing and configured to couple to a syringe. The movable handle is movable relative to the housing and operably coupled to the syringe carriage to distally advance the syringe carriage. The shaft extends distally from the housing and defines a lumen. The needle sheath extends through the lumen of the shaft, defines a lumen, and is movable relative to the shaft between an extended condition and a retracted condition. The needle extends through the lumen of the needle sheath and is configured to operably couple to a syringe coupled to the syringe carriage. A distal end of the needle is covered by the needle sheath when the needle sheath is in the extended condition and the distal end of the needle is uncovered by the needle sheath when the needle sheath is in the retracted condition.

In an aspect, the surgical instrument includes a lever operably coupled to the needle sheath and slidably coupled to the housing. The lever is configured to selectively move the needle sheath between the extended condition and the retracted condition.

In an aspect, the surgical instrument includes a seal coupled between an outer surface of the needle and an inner surface of the needle sheath forming a fluid-tight seal therebetween when the needle sheath is in the retracted position and forming a fluid seal when the needle sheath is in the extended condition. In an aspect, the fluid-tight seal is arranged such that when the needle sheath is fully extended the fluid-tight seal is distal of the needle tip and closes the needle sheath to fluid loss.

In an aspect, the surgical instrument includes a fluid port extending from the housing and in fluid communication with the lumen of the needle sheath.

In an aspect, a proximal end of the needle includes a luer lock for coupling to a distal end of the syringe.

In an aspect, the syringe carriage includes a gear rack and the movable handle is operably coupled to a pinion gear such that movement of the movable handle causes rotation of the pinion gear and linear translation of the gear rack of the syringe carriage. The movable handle and the pinion gear may be in ratchet engagement with each other.

In an aspect, the surgical instrument includes a kick-back assembly configured to bias the syringe carriage in a proximal direction. The kick-back assembly includes an adjustable kick-back lever, a kick-back stop including a tooth operably coupled to the gear rack of the syringe carriage, and a spring disposed between the adjustable kick-back lever and the kick-back stop. The adjustable kick-back lever is configured to selectively adjust the length of travel for the proximal bias back at the end of each injection.

In another aspect of the disclosure, a surgical instrument includes a housing, a syringe carriage, a syringe, a movable handle, a shaft, a needle sheath, a lever, and a needle. The syringe carriage is operably coupled to the housing and houses the syringe which stores a fluid therein. The movable handle is movable relative to the housing and is operably coupled to the syringe carriage to distally advance the syringe carriage and expel the fluid from the syringe. The shaft extends distally from the housing, and defines a lumen. The needle sheath extends through the lumen of the shaft, defines a lumen, and is movable relative to the shaft between an extended condition and a retracted condition. The lever is operably coupled to the needle sheath and is configured to move the needle sheath. The needle extends through the lumen of the needle sheath and is operably coupled to the syringe to deliver the fluid from the syringe.

In an aspect, a distal end of the needle is covered by the needle sheath when the needle sheath is in the extended condition and the distal end of the needle is uncovered by the needle sheath when the needle sheath is in the retracted condition.

In an aspect, the surgical instrument includes a seal coupled between an outer surface of the needle and an inner surface of the needle sheath forming a fluid-tight seal therebetween when the needle sheath is in the retracted position and forming a fluid seal when the needle sheath is in the extended condition. In an aspect, the fluid-tight seal is arranged such that when the needle sheath is fully extended the fluid-tight seal is distal of the needle tip and closes the needle sheath to fluid loss.

In an aspect, the surgical instrument includes a fluid port extending from the housing and in fluid communication with the lumen of the needle sheath.

In an aspect, a proximal end of the needle includes a luer lock for coupling to a distal end of the syringe.

In an aspect, the syringe carriage includes a gear rack and the movable handle is operably coupled to a pinion gear such that movement of the movable handle causes rotation of the pinion gear and linear translation of the gear rack of the syringe carriage. The movable handle and the pinion gear may be in ratchet engagement with each other.

In an aspect, the surgical instrument includes a kick-back assembly configured to bias the syringe carriage in a proximal direction. The kick-back assembly includes an adjustable kick-back lever, a kick-back stop including a tooth operably coupled to the gear rack of the syringe carriage, and a spring disposed between the adjustable kick-back lever and the kick-back stop. The adjustable kick-back lever is configured to selectively adjust the length of travel for the proximal bias back at the end of each injection.

In another aspect of the disclosure, a surgical instrument includes a housing, a syringe carriage operably coupled to the housing and configured to couple to a syringe, a movable handle movable relative to the housing and operably coupled to the syringe carriage to distally advance the syringe carriage, a shaft extending distally from the housing and defining a lumen, a needle sheath extending through the lumen of the shaft, and a kick-back assembly configured to bias the syringe carriage in a proximal direction. The needle sheath defines a lumen configured to receive a needle therethrough and is movable relative to the shaft between an extended condition and a retracted condition.

In accordance with yet another aspect of the present disclosure, a robotic surgical catheter unit includes a catheter head assembly, a microtube coupled to the catheter head assembly and a guidewire. The catheter head assembly includes a housing, a grasper tab extending from the housing, an injection needle extending distally from the housing, a retractable sheath moveable relative to the injection needle between an extended condition and a retracted condition, a check valve disposed within the housing, and a resilient member disposed within the housing and configured to bias the retractable sheath to the extended condition. The microtube is operably coupled to a proximal portion of the housing and configured to couple to a fluid supply. The guidewire is operably coupled to the retractable sheath and configured to control movement of the retractable sheath between the extended condition and the retracted condition.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

Various aspects of the disclosure are described herein below with reference to the drawings, wherein:

FIG. 1 is a side view of a surgical instrument in accordance with the disclosure;

FIG. 2 is a top, side, perspective view of the surgical instrument of FIG. 1;

FIG. 3 is a cross-sectional view of the surgical instrument of FIG. 1;

FIG. 4 is another cross-sectional view of the surgical instrument of FIG. 1;

FIG. 5 is a side view of the surgical instrument of FIG. 1 with a portion of a housing of the surgical instrument removed;

FIG. 6 is a side perspective view of the surgical instrument of FIG. 1 with a portion of the housing and parts removed;

FIG. 7 is an enlarged view of the indicated area of detail shown in FIG. 1;

FIG. 8 is a schematic view of a robotic system including a surgical robotic catheter unit in accordance with the disclosure;

FIG. 9 is a side, cross-sectional view of a catheter head assembly of the surgical robotic catheter unit of FIG. 8; and

FIG. 10 is a side view of a surgical grasping unit grasping the catheter head assembly of FIG. 9.

### DETAILED DESCRIPTION

In this description, the term “proximal” is used generally to refer to that portion of the device that is closer to a clinician, while the term “distal” is used generally to refer to that portion of the device that is farther from the clinician. Further, the term “clinician” is used generally to refer to medical personnel including doctors, nurses, and support personnel.

The disclosure describes a needle injection instrument, with a retractable sheath covering a needle, that includes a pump handle that provides limited and precise dosing with each squeeze of the handle. In use, the needle is covered by the retractable sheath as it is passed through a laparoscopic port and brought into close approximation to a target injection point. The retractable sheath may then be pulled back exposing the needle for immediate insertion into the target tissue with little risk of losing imaging elsewhere in the abdomen. The pump handle then enables the user to inject a precise amount of fluid (e.g., ICG solution) from the syringe while maintaining visual attention to the injection sight. Once the intended volume of fluid (e.g., ICG solution) is injected by number of counts of pumps of the handle, the retractable sheath is extended toward the tissue pushing the tissue off of the needle and covering the needle as it clears the tissue surface reducing the likelihood of any loss of fluid (e.g., ICG solution) into the abdomen or surgical area.

The disclosed surgical instrument 10 is designed in a manner that it can be operated with one hand, injects very small volumes of fluid at predetermined volumes for each actuation of the handle, has a shielded needle for trocar pass-through and has drip prevention features in the form of automatic drawback and a pierceable membrane at the end of the needle sheath. The needle covering (e.g., needle sheath) may be spring operated in one direction, for example distally biased to cover the needle.

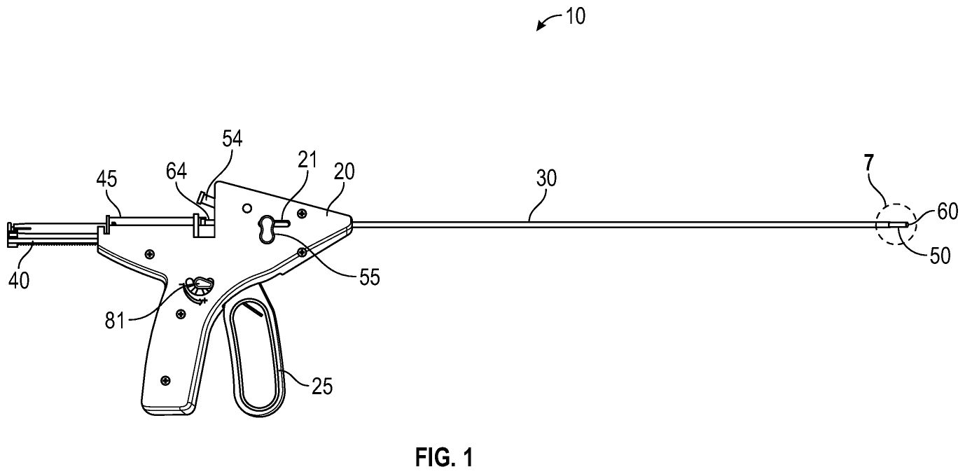

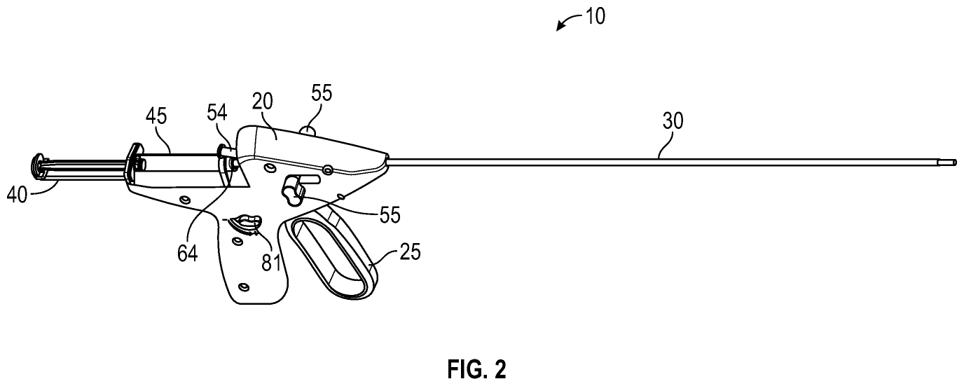

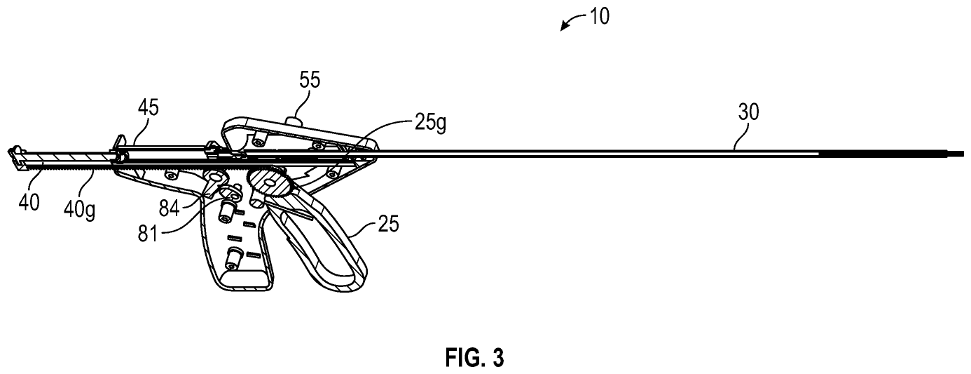

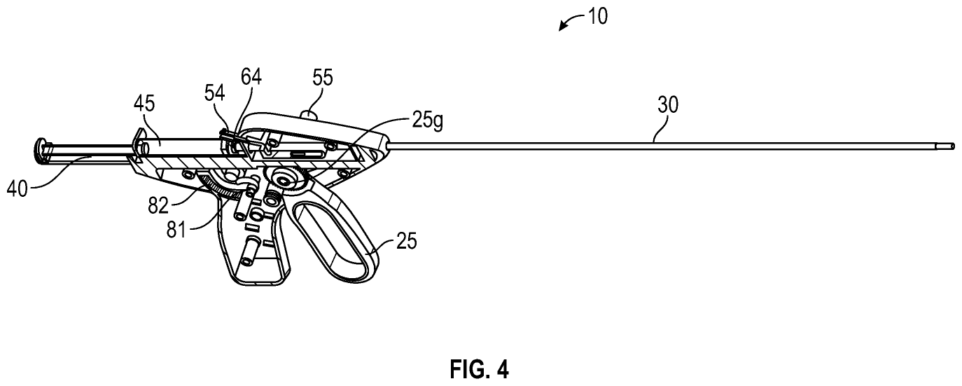

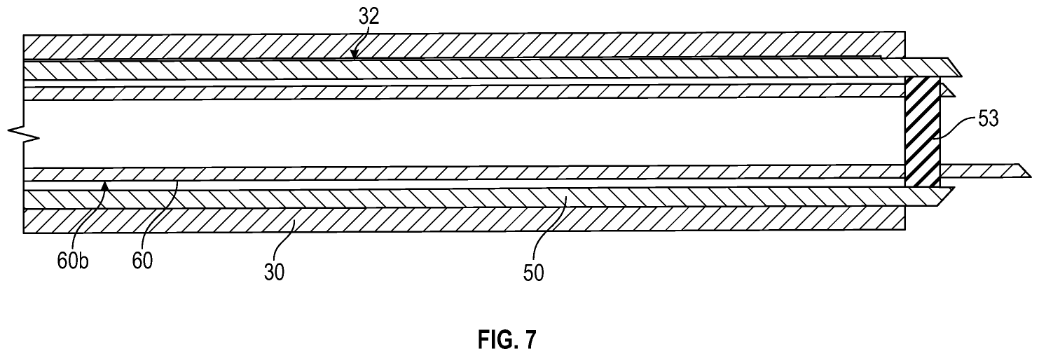

FIGS. 1-7 illustrate an exemplary surgical instrument 10 in accordance with the disclosure. The surgical instrument 10 includes a housing 20, a syringe carriage 40 configured to house a syringe 45, and a movable handle 25 operably coupled to the syringe carriage 40 to distally advance the syringe carriage 40. A shaft 30 defining a lumen 32 extends distally from the housing 20 and a needle sheath 50 extends through the lumen 32 of the shaft 30. The instrument may also include a lever 55 extending through a slot 21 defined through the housing 20 and operably coupled to the needle sheath 50 for moving the needle sheath 50 relative to the housing 20. The needle sheath 50 defines a lumen 52 and is movable relative to the shaft 30 between an extended condition and an approximated condition. A needle 60 extends through the lumen 52 of the needle sheath 50 and operably is coupled to the syringe 45 to deliver fluid from the syringe 45. A distal end 60b of the needle 60 is covered by the needle sheath 50 when the needle sheath 50 is in the extended condition and the distal end 60b of the needle 60 is uncovered by the needle sheath 50 when the needle sheath 50 is in the retracted condition.

The lever 55 is coupled to the needle sheath 50 (e.g., at a proximal portion of the needle sheath 50) and is slidable relative to the housing 20. The lever 55 is selectively slidable by a user to move the needle sheath 50 between the extended condition, where the needle sheath 50 covers the distal end 60b of the needle 60, and the retracted condition, where the needle sheath 50 does not cover the distal end 60b of the needle 60. In an aspect, the lever 55 is biased toward one direction. A seal 53 is positioned at a distal end of the needle sheath 50 which is pierceable by the needle 60 as the needle sheath 50 is moved toward the retracted condition. The seal 53 may assist in limiting fluid ingress during use and prevents fluid egress when the needle 60 is shielded by the needle sheath 50 (e.g., when in the retracted condition). The fluid-tight seal is arranged such that when the needle sheath 50 is fully extended, the fluid-tight seal is distal of the needle tip and closes the needle sheath 50 to fluid loss.

A fluid port 54 extends from the housing 20 for connection to a vent or suction device (not shown). The fluid port 54 is in fluid communication with the lumen 52 of the needle sheath 50, thereby forming a fluid path between an outer surface of the needle 60 and the inner surface of the needle sheath 50 when the needle 60 is positioned through the lumen of 52 of the needle sheath 50. The fluid port 54 may capture fluid escaping from the residual pressure in the needle 60 or the injected tissue. The surgical instrument 10 may also include a shut-off to the fluid port 54 extending from the housing and in communication with the lumen 52 of the needle sheath 50 such that when the needle sheath 50 is retracted proximally the fluid port 54 is closed. For example, a proximal end of the needle sheath 50 may end in a compartment and when positioned all the way proximally (e.g., in the retracted-most position) blocks the fluid port 54 from the compartment to the luer of the fluid port 54 to prevent the flow of fluid therethrough.

A proximal end of the needle 60 includes a luer lock 64 for coupling to a distal end of a syringe 45 secured to the syringe carriage 40. The syringe 45 is configured to house fluid (e.g., drugs, imaging fluids, etc.) for delivery through the needle 60 into a target site upon actuation of the syringe carriage 40 to which the syringe 45 is coupled. In particular, the syringe carriage 40 is coupled to the movable handle 25 in a rack and pinion arrangement such that when the movable handle 25 is moved toward a fixed handle (not shown) of the housing 20, or otherwise actuated, the syringe carriage 40 moves a plunger (not shown) of the syringe 45 thereby causing fluid to expel from the syringe 45 and through the needle 60. Each actuation of the movable handle 25 causes a precise predetermined dose of fluid to be delivered from the syringe 45.

The syringe carriage 40 includes a gear rack 40g defined along its bottom surface which is meshingly engaged with a pinion gear 25g rotatably controlled by movement of the movable handle 25. In one aspect, the pinion gear 25g is coupled to the movable handle 25 in a ratchet engagement. Movement of the movable handle 25 causes rotation of the pinion gear 25g which, in turn, causes longitudinal translation of the gear rack 40g.

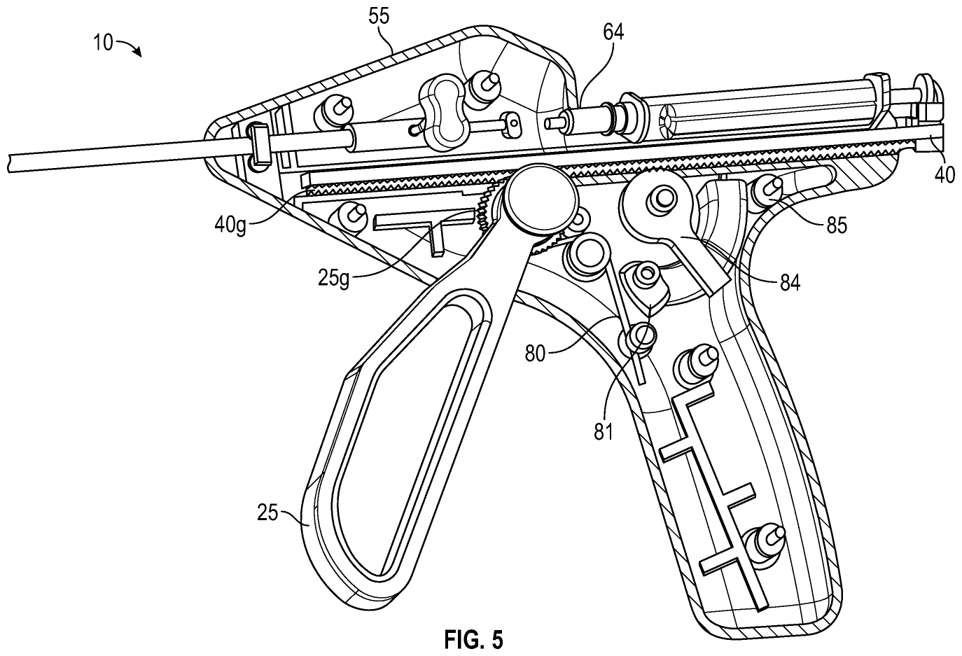

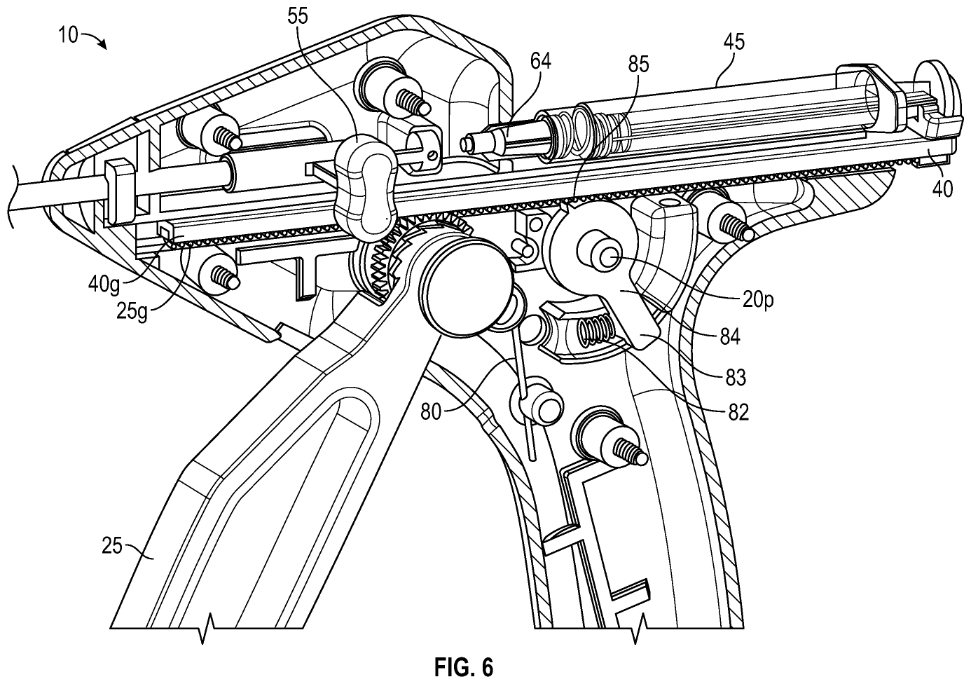

With particular reference to FIGS. 5 and 6, the surgical instrument 10 may also include a kick-back assembly 80 configured to bias the syringe carriage 40 in a proximal direction or otherwise prevent drippage of fluid from the distal end 60b of the needle 60 (e.g., after fluid is delivered therefrom). The kick-back assembly 80 may be selectively adjustable (e.g., by a user) to adjust the distance the kick-back withdraws the syringe carriage 40. The kick-back assembly 80 includes an adjustable kick-back lever 81 extending through the housing 20 and selectively adjustable by a user, a kick-back stop 84 rotatable relative to the housing 20 about a pivot point 20p, and a spring 82. The kick-back stop 84 includes an arm 83 on one side of the pivot point 20p and a tooth 85 on the other side of the pivot point 20p. The spring 82 is positioned between the kick-back lever 81 (e.g., along a camming surface of the kick-back lever 81) and the arm 83 of the kick-back stop 84 and provides a biasing force against the arm 83 of the kick-back stop 84. The tooth 85 of the kick-back stop 84 is operably coupled to the gear rack 40g of the syringe carriage 40. The adjustable kick-back lever 81 is configured to selectively adjust the travel of the spring 82 against the arm 83 of the kick-back stop 84 to, in turn, adjust the distance the syringe carriage 40 can be withdrawn proximally.

The surgical instrument 10 is illustrated as a manually actuatable surgical instrument, but it is appreciated that surgical instrument 10 may be an electrically powered surgical instrument including an electrically powered handle assembly that may support one or more batteries (not shown). It is envisioned that the disclosed aspects could also be incorporated into a surgical instrument that is configured for use with a robotic system that does not include a handle assembly, or to a surgical instrument including a manually actuated handle assembly.

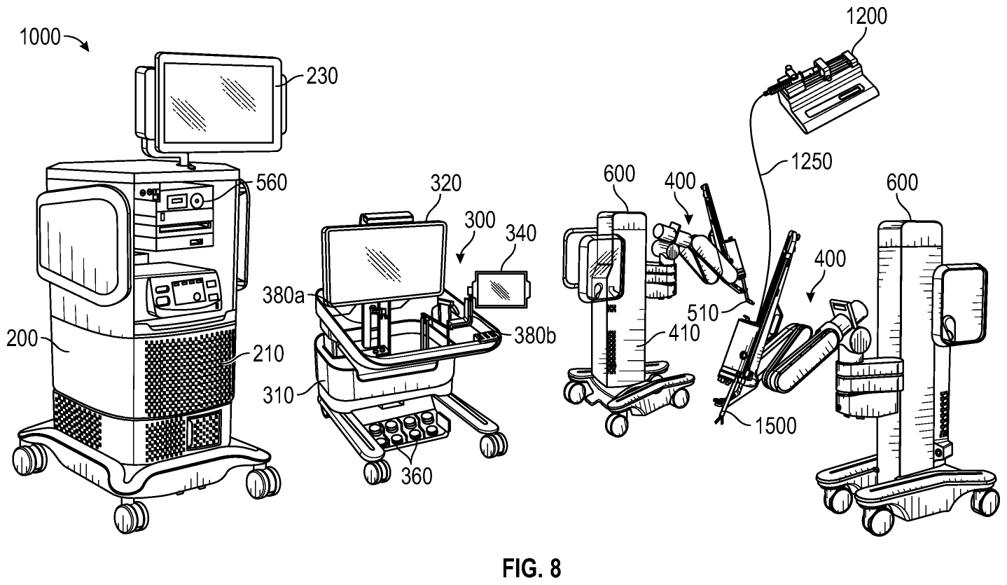

Turning to FIGS. 8-10, a surgical robotic system 1000 with a robotic surgical catheter unit having an injection needle and a retractable sheath for covering the injection needle will now be described. The surgical robotic system 1000 includes a surgical console, a control tower, and one or more movable carts having a surgical robotic arm coupled to a setup arm. The surgical console receives user input through one or more interface devices, which are interpreted by the control tower as movement commands for moving the surgical robotic arm. The surgical robotic arm includes a controller, which is configured to process the movement command and to generate a torque command for activating one or more actuators of the robotic arm, which would, in turn, move the robotic arm in response to the movement command.

With reference to FIG. 8, a surgical robotic system 1000 includes a control tower 200, which is connected to all of the components of the surgical robotic system 1000 including a surgical console 300 and one or more robotic arms 400. Each of the robotic arms 400 includes a surgical instrument, for example, a stapler, grasper, forceps, catheter head unit 1500, or any other surgical instrument removably coupled thereto. Each of the robotic arms 400 is also coupled to a movable cart 600.

The surgical instrument is configured for use during minimally invasive surgical procedures. In embodiments, the surgical instrument may be configured for open surgical procedures. In embodiments, the surgical instrument may be an endoscope, such as an endoscopic camera 510, configured to provide a video feed for the user. In further embodiments, the surgical instrument may be an electrosurgical forceps configured to seal tissue by compressing tissue between jaw members and applying electrosurgical current thereto. In yet further embodiments, the surgical instrument may be a surgical stapler including a pair of jaws configured to grasp and clamp tissue while deploying a plurality of tissue fasteners, e.g., staples, and cutting stapled tissue.

One of the robotic arms 400 may include the endoscopic camera 510 configured to capture video of the surgical site. The endoscopic camera 510 may be a stereoscopic endoscope configured to capture two side-by-side (i.e., left and right) images of the surgical site to produce a video stream of the surgical scene. The endoscopic camera 510 is coupled to a video processing device 560, which may be disposed within the control tower 200. The video processing device 560 may be any computing device as described below configured to receive the video feed from the endoscopic camera 510, perform image processing based on the depth estimating algorithms, and output the processed video stream.

The surgical console 300 includes a first display 320, which displays a video feed of the surgical site provided by camera 510 disposed on the robotic arms 400, and a second display 340, which displays a user interface for controlling the surgical robotic system 1000. The first and second displays 320 and 340 may be touchscreens allowing for displaying various graphical user inputs.

The surgical console 300 also includes a plurality of user interface devices, such as foot pedals 360 and a pair of handle controllers 380a and 380b which are used by a user to remotely control robotic arms 400 and any surgical instrument(s) coupled thereto.

The control tower 200 includes a display 230, which may be a touchscreen, and outputs on the graphical user interfaces (GUIs). The control tower 200 also acts as an interface between the surgical console 300 and one or more robotic arms 400. In particular, the control tower 200 is configured to control the robotic arms 400, such as to move the robotic arms 400 and the corresponding surgical instrument, based on a set of programmable instructions and/or input commands from the surgical console 300, in such a way that robotic arms 400 and the surgical instrument execute a desired movement sequence in response to input from the foot pedals 360 and the handle controllers 380a and 380b.

Each of the control tower 200, the surgical console 300, and the robotic arm 400 includes a respective computer 210, 310, 410. The computers 210, 310, 410 are interconnected to each other using any suitable communication network based on wired or wireless communication protocols. The term “network,” whether plural or singular, as used herein, denotes a data network, including, but not limited to, the Internet, Intranet, a wide area network, or a local area networks, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-2003 standard for wireless personal area networks (WPANs)).

The computers 210, 310, 410 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

The surgical robotic catheter unit 1500 can be operated remotely and via a user interface of a robotic console controlled by a surgeon. The user interface is customizable per the user's needs. The robotic catheter unit 1500 is configured to inject very small volumes of fluid (e.g., ICG), has a shielded needle for trocar pass through, and has drip prevention features, for example in the form of a check valve, and a pierceable membrane at the end of its needle sheath. The needle covering may be spring operated in one direction (for example, distally).

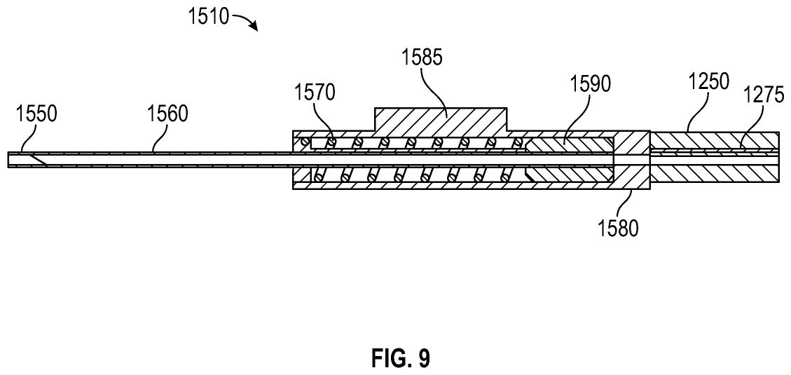

The robotic catheter unit 1500 includes a catheter head assembly 1510 (FIG. 9) and is coupled to a syringe pump 1200 (FIG. 8) via a double lumen micro tube 1250. The catheter head assembly 1510 includes a housing 1580 with a grasper tab 1582 extending outwardly from a side of the housing 1580 and a retractable sheath 1550 extending distally from the housing 1580 and movable relative to the housing 1580. A distal portion of a guidewire 1275 is operably coupled to the retractable sheath 1550 such that when the guidewire 1275 is pulled proximally, the retractable sheath 1150 also moves proximally (e.g., retracted). An injection needle 1560 is in fluid communication with the micro tube 1250, extends distally from the housing 1580 and may be at least partially disposed within the housing 1580. A resilient member 1570 (e.g., a spring mechanism) and a check valve 1590 are disposed at least partially within the housing 1580. The resilient member 1570 is configured to bias the retractable sheath 1550 distally, that is, to a position where the retractable sheath 1550 covers at least a distal end of the injection needle 1560. The retractable sheath 1550 is also connected to a guidewire 1275, which when pulled proximally, in turn pulls the proximal side of the retractable sheath 1550 relative to the injection needle 1560 to uncover the distal end of the injection needle 1560.

The check valve 1590 is placed in the catheter head assembly 1510 so that it can open when sufficient pressure is applied otherwise it will not allow fluid to pass through, thereby preventing any dripping of fluid from the catheter head assembly 1510 (e.g., to inside the abdominal body cavity).

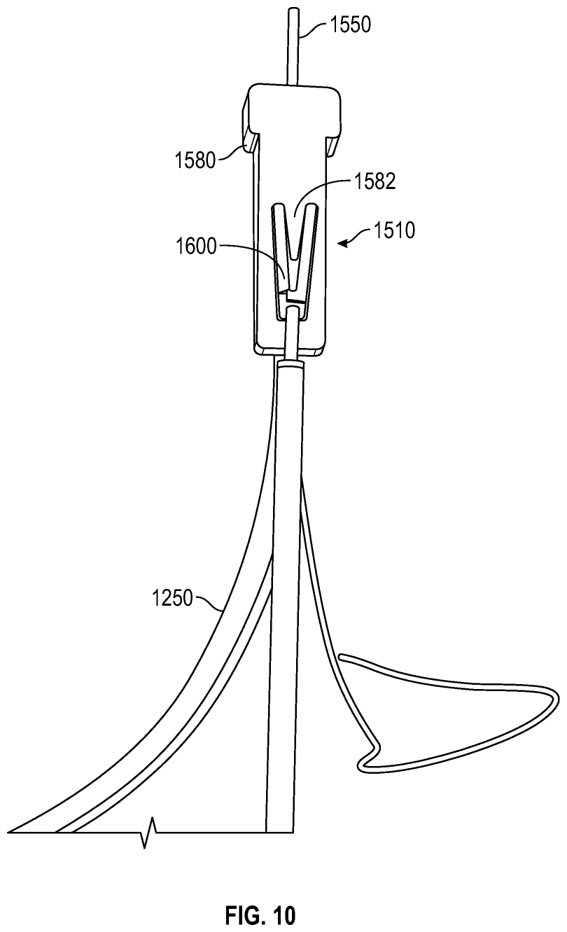

The grasper tab 1582 of the catheter head assembly 1510 is dimensioned such that it can be easily held by a grasper unit (e.g., grasper unit 1600 in FIG. 10). In particular, the shape form and size of the grasper tab 1582 is configured to be easily grasped by a variety of robotic grasper units, for example, a grasping device coupled to a robotic arm or a hand-held grasping device held by an operator.

A distal portion of the double lumen micro tube 1250 is connected to a proximal portion of the housing 1580 of the catheter head assembly 1510 and directly to the injection needle 1560. A proximal end the double lumen micro tube 1250 is configured to be connected to a fluid supply, for example a syringe connected to a syringe pump 1200 (FIG. 8). One tube of the double lumen micro tube 1250 is used to pass the fluid from a syringe in the syringe pump 1200 to the injection needle 1560. The other tube of the double lumen micro tube 1250 may be used to carry the guidewire 1275 for controlling movement of the retractable sheath 1550.

The syringe pump 1200 and mechanisms for controlling the positioning of the guidewire 1275 are coupled to, and controlled by, the robotic control tower 200 for providing precise dosage delivery by the syringe pump 1200 and for providing precise control of the movement and position of the guidewire 1275 to control the position of the retractable sheath 1550 over the injection needle 1560. At the surgical console 300, the user interface provides data corresponding to the position of the retractable sheath 1550, the injection pressure, and the total injected volume for the procedure and for the current injection (as some injectables may have a limited total dose).

In an aspect, the injection pressures are derived from a force on the syringe pump 1200 or, for more precision, an inline sensor (not shown) attached to the micro tube 1250 (e.g., at the syringe pump 1200 or at any portion of the catheter head unit 1500). The injection pressure may be further refined by subtracting the opening pressure of a valve (e.g., check valve 1590) through which the injectable passes. The opening pressure may be determined dynamically by the system or as predetermined calibration value.

Automated feedback on the pressure provides a more precise determination of the injection location. For example, the algorithms may be based on an injection rate and back-pressure values since the volume of fluid is being absorbed into the tissue particularly avoiding vessels where fluid back-pressure will be particularly low or solid tissue, where the fluid pressure will rise quickly. In other areas, the injection volume and/or back-pressure may be tied to the camera view providing correlation of volume, pressure, and observed tissue bulge.

During operation, the catheter head assembly 1510 can be inserted into the abdominal body cavity by a small push force through a 10 mm trocar unit. While inserting the catheter head assembly 1510, the retractable sheath 1550 is covering the injection needle 1560 so that it may be safely placed inside the body cavity without damaging trocar pressure seals and without injuring internal organs. The catheter head assembly 1510 can be navigated to the intended surgical area and positioned within the body cavity by a robotic or surgical grasper unit (e.g., by a surgical/robotic grasper inserted into another trocar unit different from the one in which the catheter head assembly 1510 is inserted through).

Once the catheter head assembly 1510 reaches the injection target, tissue, or vessel, the retractable sheath 1550 can be pulled back by the guidewire 1275 which will expose a sharp distal end of the injection needle 1560. The injection needle 1560 is further guided into the tissue by a grasper unit 1600 (FIG. 10) grasping the grasper tab 1582 of the catheter head assembly 1510. Once the injection needle 1560 is inserted, the syringe pump 1200 can be operated to pass the required amount of solution to the intended tissue. Solutions may include, for example, solutions used for tumescent surgery, local injection of fluorophores (ICG) or anesthetic solutions, or any other injectable. Once the injection of fluid is complete, the injection needle 1560 can be pulled back by the grasper unit 1600 grasping the grasper tab 1582 and the retractable sheath 1550 can be controlled and/or biased to cover the injection needle 1560. Additionally, or alternatively, the tissue may be pushed from the injection needle 1560 by deploying the retractable sheath 1550 and/or enabling the deployment of the retractable sheath 1550 (e.g., by releasing the guidewire 1275). The check valve 1590 will operate based on the set pressure and will not allow fluid coming from the micro tube 1250 to drip out of the tip of the injection needle 1560.

The disclosed catheter unit assists to reduce ICG lost from the needle and does not reduce the quality of the NIR imaging camera and system, thereby improving its ability to mark or map the tissue. Additionally, the design of the catheter unit reduces safety risks to users and inadvertent tissue sticks inside the patient. Additionally, the disclosed catheter unit reduces the risk of damaging laparoscopic ports that contain pressure seals inside when the catheter unit is passed through the port. The disclosed catheter unit is compatible with robotic platforms and is easily customizable to meet user needs by using a syringe pump and an ECU unit. This system can be operated remotely and improves the current standard of care for catheter injection needles. The total amount of fluid injected over time may be monitored by the system to assure safe and accurate dosing. The back-pressure provides automated feedback to ensure that the injected solution is going into the correct location, not solid tissue (high pressure) or a vessel (low pressure).

Persons skilled in the art will understand that the devices and methods specifically described herein and illustrated in the accompanying drawings are non-limiting exemplary embodiments. It is envisioned that the elements and features illustrated or described in connection with one exemplary embodiment may be combined with the elements and features of another without departing from the scope of the disclosure. As well, one skilled in the art will appreciate further features and advantages of the disclosure based on the above-described embodiments. Accordingly, the disclosure is not to be limited by what has been particularly shown and described, except as indicated by the appended claims.

What is claimed is:

1. A surgical instrument comprising:
a housing;
a syringe carriage operably coupled to the housing and configured to couple to a syringe;
a movable handle movable relative to the housing and operably coupled to the syringe carriage to distally advance the syringe carriage;
a shaft extending distally from the housing and defining a lumen;
a needle sheath extending through the lumen of the shaft, the needle sheath defining a lumen and being movable relative to the shaft between an extended condition and a retracted condition;
a needle extending through the lumen of the needle sheath and configured to operably couple to a syringe coupled to the syringe carriage, wherein a distal end of the needle is covered by the needle sheath when the needle sheath is in the extended condition and the distal end of the needle is uncovered by the needle sheath when the needle sheath is in the retracted condition; and
a kick-back assembly configured to bias the syringe carriage in a proximal direction.

2. The surgical instrument of claim 1, further comprising a lever operably coupled to the needle sheath and slidably coupled to the housing, the lever configured to selectively move the needle sheath between the extended condition and the retracted condition.

3. The surgical instrument of claim 1, further comprising a seal coupled to an inner surface of the needle sheath and forming a fluid-tight seal between the needle sheath and an outer surface of the needle when the needle sheath is in the retracted condition and forming a seal when the needle sheath is in the extended condition.

4. The surgical instrument of claim 1, further comprising a fluid port extending from the housing and in fluid communication with the lumen of the needle sheath.

5. The surgical instrument of claim 1, wherein a proximal end of the needle includes a luer lock for coupling to a distal end of the syringe.

6. The surgical instrument of claim 1, wherein the syringe carriage includes a gear rack and the movable handle is operably coupled to a pinion gear such that movement of the movable handle causes rotation of the pinion gear and linear translation of the gear rack of the syringe carriage.

7. The surgical instrument of claim 6, wherein the movable handle and the pinion gear are in ratchet engagement with each other.

8. The surgical instrument of claim 1, wherein each actuation of the movable handle causes a specific dose to be delivered from the syringe.

9. The surgical instrument of claim 6, wherein the kick-back assembly includes an adjustable kick-back lever, a kick-back stop including a tooth operably coupled to the gear rack of the syringe carriage, and a spring disposed between the adjustable kick-back lever and the kick-back stop.

10. The surgical instrument of claim 9, wherein the adjustable kick-back lever is configured to selectively adjust a pull-back distance that the rack is moved proximally at the end of each injection stroke.

11. A surgical instrument comprising:
a housing;
a syringe carriage operably coupled to the housing;
a syringe storing a fluid and operably coupled to the syringe carriage;
a movable handle movable relative to the housing and operably coupled to the syringe carriage to distally advance the syringe carriage and expel the fluid from the syringe;
a shaft extending distally from the housing and defining a lumen;
a needle sheath extending through the lumen of the shaft, the needle sheath defining a lumen and being movable relative to the shaft between an extended condition and a retracted condition;
a lever operably coupled to the needle sheath and configured to move the needle sheath;
a needle extending through the lumen of the needle sheath and operably coupled to the syringe to deliver the fluid from the syringe; and
a kick-back assembly configured to bias the syringe carriage in a proximal direction.

12. The surgical instrument of claim 11, wherein a distal end of the needle is covered by the needle sheath when the needle sheath is in the extended condition and the distal end of the needle is uncovered by the needle sheath when the needle sheath is in the retracted condition.

13. The surgical instrument of claim 11, further comprising a seal coupled to an inner surface of the needle sheath and forming a fluid-tight seal between the needle sheath and an outer surface of the needle when the needle sheath is in the retracted condition and forming a seal when the needle sheath is in the extended condition.

14. The surgical instrument of claim 11, further comprising a fluid port extending from the housing and in fluid communication with the lumen of the needle sheath.

15. The surgical instrument of claim 11, wherein a proximal end of the needle includes a luer lock for coupling to a distal end of the syringe.

16. The surgical instrument of claim 11, wherein the syringe carriage includes a gear rack and the movable handle is operably coupled to a pinion gear such that movement of the movable handle causes rotation of the pinion gear and linear translation of the gear rack of the syringe carriage.

17. The surgical instrument of claim 16, wherein the movable handle and the pinion gear are in ratchet engagement with each other.

18. The surgical instrument of claim 11, wherein each actuation of the movable handle causes a specific dose to be delivered from the syringe.

19. The surgical instrument of claim 16, wherein the kick-back assembly includes an adjustable kick-back lever, a kick-back stop including a tooth operably coupled to the gear rack of the syringe carriage, and a spring disposed between the adjustable kick-back lever and the kick-back stop.

20. A robotic surgical catheter unit comprising:
a catheter head assembly including:
a housing and a grasper tab extending from the housing;
an injection needle extending distally from the housing;
a retractable sheath moveable relative to the injection needle between an extended condition and a retracted condition;
a check valve disposed within the housing; and
a resilient member disposed within the housing and configured to bias the retractable sheath to the extended condition;
a microtube operably coupled to a proximal portion of the housing and configured to couple to a fluid supply; and
a guidewire operably coupled to the retractable sheath and configured to control movement of the retractable sheath between the extended condition and the retracted condition.
